(19) United States
(12) Patent Application Publication
(10) Pub. No.: US 2010/0331708 A1
(43) Pub. Date: Dec. 30, 2010

(54) MONITORING CARDIOVASCULAR CONDITIONS USING SIGNAL TRANSIT TIMES

(75) Inventor: Feras Hatib, Irvine, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/822,122

(22) Filed: Jun. 23, 2010

Related U.S. Application Data
(60) Provisional application No. 61/221,238, filed on Jun. 29, 2009.

(51) Int. Cl. A61B 5/02

(52) U.S. Cl. 600/481

(57) ABSTRACT

Methods for monitoring cardiovascular conditions, i.e., hyperdynamic circulation, vasodilation, vasoconstriction, or central-to-peripheral arterial pressure decoupling conditions are described. These methods involve measuring a central signal proportional to or a function of the subject's heart activity and a peripheral signal proportional to or a function of a signal related to the central signal. Then calculating a time difference between features in the central and peripheral signals representing the same heart event. The cardiovascular condition is indicated if the time difference is greater or lower than a threshold value, if the time difference is greater or lower than a threshold value over a specified period of time, or if there is a significant statistical change in the times over the specified time period. These methods can alert a user that a subject is experiencing the cardiovascular condition, which can enable a clinician to appropriately provide treatment to the subject.

### CLAIM OF PRIORITY UNDER 35 U.S.C. §119

The application claims the benefit of U.S. Provisional Application No. 61/221,238 filed Jun. 29, 2009, entitled “Monitoring Cardiovascular Conditions Using Signal Transit Times” and assigned to the assignee hereof and hereby incorporated by reference in its entirety.

### BACKGROUND

Indicators such as stroke volume (SV), cardiac output (CO), end-diastolic volume, ejection fraction, stroke volume variation (SVV), pulse pressure variation (PPV), and systolic pressure variations (SPV), among others, are important not only for diagnosis of disease, but also for “real-time,” i.e., continual, monitoring of clinically significant changes in a subject. For example, health care providers are interested in changes in preload dependence, fluid responsiveness, or volume responsiveness as well as, for example, central-to-peripheral decoupling in both human and animal subjects. Few hospitals are therefore without some form of equipment to monitor one or more cardiac indicators in an effort to provide a warning that one or more of the indicated changes are occurring in a subject. Many techniques, including invasive techniques, non-invasive techniques, and combinations thereof, are in use and even more have been proposed in the literature.

### SUMMARY

Methods for monitoring a cardiovascular condition in a subject are described. The methods involve measuring a central signal proportional to or a function of the subject's heart activity and a peripheral signal proportional to or a function of a signal related to the central signal. A time difference between signal features representing the same heart events for the central signal and the peripheral signal is then calculated and the cardiac condition is indicated if the time difference is greater than a threshold value.

Additional methods for monitoring a cardiovascular condition in a subject involve measuring a central signal proportional to or a function of the subject's heart activity and a peripheral signal proportional to or a function of a signal related to the central signal. A time difference between signal features representing the same heart event for the central signal and the peripheral signal is then calculated and the cardiovascular condition is indicated if there is a significant statistical change in the time difference over a specified time period.

### DESCRIPTION OF DRAWINGS

FIG. 1 shows simultaneously recorded pressure waveforms in the ascending aorta (Aortic), femoral artery (Femoral), and radial artery (Radial) in a porcine animal model during normal hemodynamic conditions.

FIG. 2 shows simultaneously recorded pressure waveforms in the ascending aorta (Aortic), femoral artery (Femoral), and radial artery (Radial) in a porcine animal model during Endotoxin shock (septic shock) resuscitated with large amounts of fluids.

FIG. 3 shows a flow chart illustrating an example of logic for monitoring a cardiovascular condition in a subject using changes in time difference between analogous heart events.

FIG. 4 shows a flow chart illustrating an example of logic for monitoring a cardiovascular condition in a subject using significant statistical changes in the time difference between analogous heart events over a specified time period.

FIG. 5 shows central (ECG) and peripheral radial pressure signals with the time difference between an analogous heart event indicated.

FIG. 6 shows central aortic pressure and peripheral radial pressure signals with the time difference between an analogous heart event indicated.

FIG. 7 is a block diagram showing the main components of a system to implement the methods described herein.

### DETAILED DESCRIPTION

Methods for monitoring cardiac conditions, i.e., central-to-peripheral arterial pressure decoupling, hyperdynamic conditions, vasodilation, or vasoconstriction, are described. These methods involve measuring a central signal proportional to or a function of the subject's heart activity and a peripheral signal proportional to or a function of a signal related to the central signal. Then calculating a time difference between features in the central and peripheral signals representing the same heart event, e.g., if pressure is measured, the time difference between the pressure maximum of an identified heart beat cycle and the same pressure maximum as measured at a peripheral location. The cardiovascular condition is indicated if the time difference is greater or lower than a threshold value. The time difference can be monitored for a specified period of time, with significant statistical changes in the times over the specified time period also indicating the occurrence of the cardiovascular condition. These methods can alert a user that a subject is experiencing the cardiovascular condition, which can enable a clinician to appropriately provide treatment to the subject.

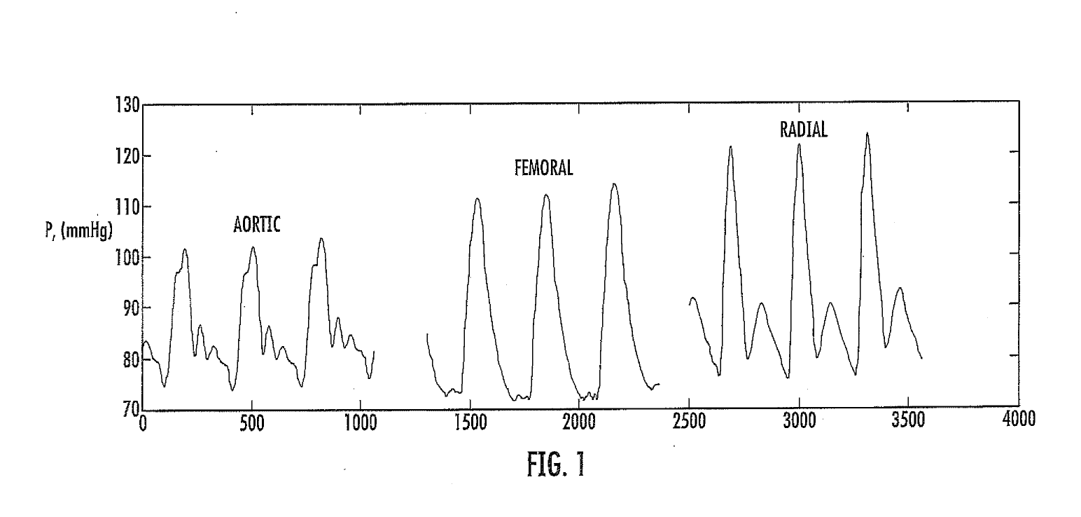

As used herein, the phrases hyperdynamic and vasodilation mean a condition in which peripheral arterial pressure and flow are decoupled from the central aortic pressure and flow, and the term peripheral arteries is intended to mean arteries located away from the heart, e.g., radial, femoral, or brachial arteries. Decoupled arterial pressure means that the normal relationship between peripheral arterial and central arterial pressure is not valid and the peripheral arterial pressure can not be used to determine the central arterial pressure. This also includes conditions in which the peripheral arterial pressure is not proportional or is not a function of the central aortic pressure. Under normal hemodynamic conditions, blood pressure increases the further away from the heart the measurement is taken. Such a pressure increase is shown in FIG. 1, i.e., the amplitude of a pressure wave measured at radial arteries is greater than the pressure measured at the femoral artery, which in turn is greater than the aortic pressure. These differences in pressure are related to wave reflection, i.e., pressure is amplified toward the periphery.

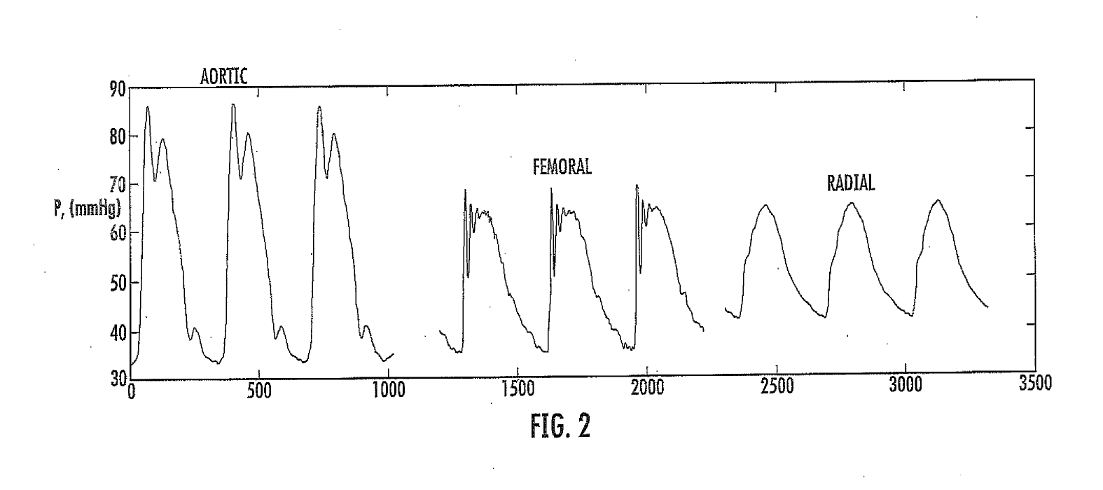

This normal hemodynamic relationship of pressures, i.e., an increase in pressure away from the heart, is often relied upon in medical diagnosis. However, under hyperdynamic/vasodilation conditions, this relationship can become inverted with the arterial pressure becoming lower than the central aortic pressure. This reversal has been attributed, for example, to arterial tone in the peripheral vessels, which is suggested to impact the wave reflections discussed above. Such a hyperdynamic condition is shown in FIG. 2, i.e., the amplitude of a pressure wave measured at radial arteries is lower than the pressure measured as the femoral artery, which in turn is lower than the aortic pressure. Drugs that dilate small peripheral arteries (e.g., nitrates, ACE inhibitors, and calcium inhibitors) are thought to contribute to hyperdynamic conditions. These types of severe vasodilatory conditions are also often observed in situations right after cardiopulmonary bypass (coronary bypass), in which the radial arterial pressure underestimates the pressure in the aorta. Substantial central-to-peripheral pressure differences, where the peripheral arterial pressure underestimates the central aortic pressure, are usually observed in patients with severe sepsis who are treated with large amount of fluids, leading to severe vasodilation. Very similar conditions are also observed in patients with end-stage liver disease. As will be well appreciated by those of skill in the art, certain treatments for subjects in normal hemodynamic conditions will be approached differently than for subjects in hyperdynamic conditions. Thus, the presently disclosed methods detect cardiovascular conditions such as central-to-peripheral arterial pressure decoupling, hyperdynamic conditions, vasodilation, and vasoconstriction in a subject, if present.

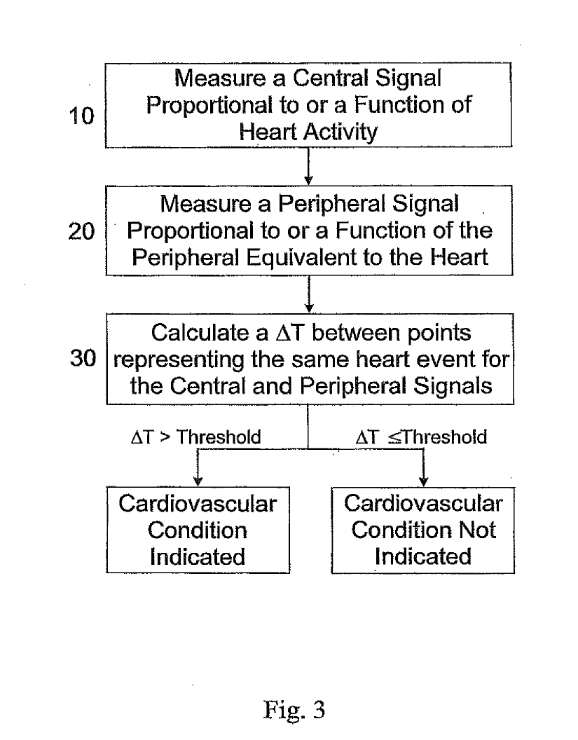

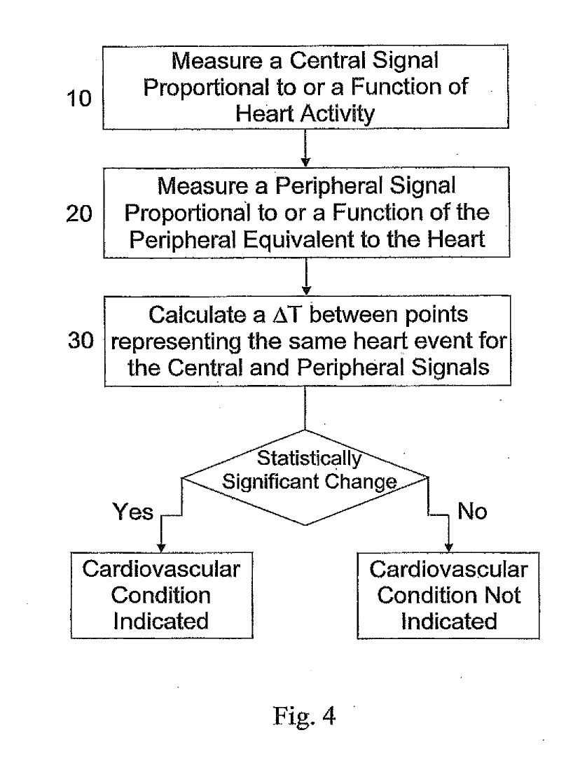

A first method for monitoring a cardiovascular condition in a subject is shown as a flow chart in FIG. 3 and involves measuring a central signal proportional to or a function of the subject's heart activity (10), and measuring a peripheral signal proportional to or a function of a peripheral equivalent to the central signal (20). Next a time difference between signal features representing the same heart events in the central signal and the peripheral signal is calculated (30). The cardiovascular condition is indicated if the time difference is greater than a threshold value or is the time difference is greater than a threshold value for a specified time period. In a further method (shown as a flow chart in FIG. 4), the cardiovascular condition is indicated if there is a significant statistical change in the time difference over a specified time period.

As used herein, the phrase central signal proportional to or a function of the subject's heart activity is used to indicate a signal related to, e.g., proportional to, derived from, or a function of, cardiac output as measured at or near the subject's heart. Examples of such signals include, but are not limited to, aortic pressure, aortic flow, pulse oximetry waveforms (for example from a central artery during invasive procedures), reflection oximetry (for example from a carotid artery, or from any central artery during invasive procedures), transthoracic bioimpedance waveforms, impedance plethysmography waveforms, electrocardiogram (ECG), ultrasound, heart sounds, and Doppler waveforms. The central signal proportional to or a function of the subject's heart activity can be directly or indirectly monitored. Examples of invasive techniques include catheter-mounted pressure transducers, catheter-mounted flow meters, and thermodilution techniques. A subject's central aortic pressure can be directly monitored, for example, with one or more pressure transducers introduced into the aorta. For direct measurement, a pressure transducer can be, for example, positioned in one or more of the subject's aortic arch, ascending aorta thoracic aorta, abdominal aorta, or the carotid artery. Other pressure meters and locations for their placement are known to those of skill in the art. Examples of non-invasive techniques include central bioimpedence plethysmography, non-invasive tonometry, ultrasound, heart sounds, and pulse/reflection oximetry. Other signals proportional to, derived from, or a function of, cardiac output as measured at or near the subject's heart and methods for their measurement are known to those of skill in the art.

The peripheral signal proportional to or a function of a signal related to the first signal is a signal related to, e.g., proportional to, derived from, or a function of, cardiac output (i.e., the first signal) as measured at a peripheral position. Examples of such signals include, but are not limited to, peripheral pressure, peripheral flow, pulse oximetry waveforms, bioimpedance plethysmography waveforms, ultrasound, tonometry, and Doppler waveforms from peripheral arteries (e.g., femoral, brachial, or radial). That the peripheral signal is a signal related to the first signal is intended to indicate that the signals are related such that features of the signals can be directly compared. Different types of signals can be measured for use with the methods described herein as long as features of the signals can be directly compared, e.g., maxima or minima that provide similar time measurements regardless of the particular measurement technique employed. By peripheral position is meant a signal measured at any point in the arterial tree located away from the subject's heart, e.g., radial, femoral, or brachial. The peripheral signal proportional to or a function of a signal related to the first signal can be measured either invasively or non-invasively. If invasive instruments are used, then any peripheral artery is a possible measurement point. For example, a subject's peripheral arterial pressure can be directly monitored with one or more pressure transducers introduced into one or more radial, brachial, or femoral vessels. Other invasive instruments and locations for their placement are known to those of skill in the art. Placement of non-invasive transducers will typically be dictated by the instruments themselves, e.g., finger cuffs, upper arm pressure cuffs, earlobe clamps, and tonometry-based pressure transducers. For example, a subject's peripheral arterial pressure can be measured by one or more of central bioimpedence plethysmography, non-invasive tonometry, ultrasound, cuff blood pressure, or pulse oximetry. Other non-invasive instruments and methods for their use are known to those of skill in the art. Regardless of the specific instrument or measurement used, the data obtained will ultimately yield an electric signal corresponding to (e.g., proportional to, derived from, or a function of,) cardiac output.

Examples of combinations of central and peripheral signals that are useful with the methods as described herein include aortic pressure (central signal) and peripheral pressure (peripheral signal), aortic flow (central signal) and peripheral flow (peripheral signal).

The features of the signals used in the methods as described herein, i.e., the features in the central and peripheral signals proportional to or a function of the subject's heart activity between which a time difference will be calculated, relate to signal features that can be measured with respect to time. For example, if pressure is measured, the minima or maxima features of the pressure signal occur at identifiable times in the signal. Further examples of such features include heart beat starting time, pressure or flow minima/maxima times, time of onset of the systolic portion of a heartbeat cycle, time of end of the systolic portion of a of a heartbeat cycle, time of onset of the diastolic portion of a heartbeat cycle, and the measured time point for the dichrotic notch.

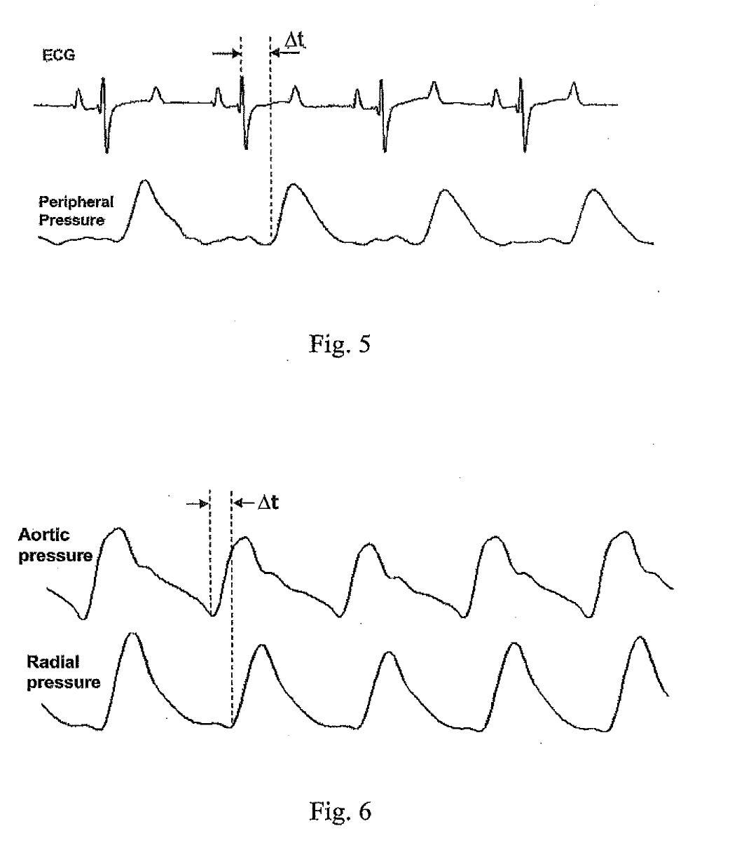

Calculating a time difference between signal features representing the same heart events for the central signal and the peripheral signal can be accomplished using methods well known to those of skill in the art. Once the signal features are identified, also by methods well known to those of skill in the art, the determined time values are simply subtracted from each other. Similarly, monitoring the change in such time differences for statistical significance can be accomplished using statistical methods well known to those of skill in the art. FIG. 5 shows a central signal (electrocardiograph (ECG)) and peripheral signal (arterial pressure measured in the radial artery) aligned in time with analogous signal features indicated with dashed lines. In FIG. 5, the time difference (Δt) is the difference in time between the dashed lines. Further, FIG. 6 shows a central signal (central aortic pressure) and a peripheral signal (peripheral radial arterial pressure) with the time difference (Δt) between analogous heart events indicated.

A cardiovascular condition such as, for example, central-to-peripheral arterial pressure decoupling, is indicated in the methods as described herein if the time difference (i.e., propagation time or transit time) between a feature in the central signal and the analogous feature in the peripheral signal is greater than (or less than) a threshold value. There is naturally some difference (usually small) between central and peripheral signals simply due to the amount of time a cardiac output signal to be realized at a peripheral location. Among other reasons known to those of skill in the art, this time lag is due to factors such as arterial compliance and wave reflections. Examples of threshold values useful with the methods as described herein include 150 milliseconds or greater, 160 milliseconds or greater, 170 milliseconds or greater, 180 milliseconds or greater, 190 milliseconds or greater, 200 milliseconds or greater, 210 milliseconds or greater, and 220 milliseconds or greater. Additionally, a cardiac condition can be indicated if the time difference is greater than (or less than) a threshold value for a specified time period. Examples of specified time periods useful with the methods as described herein include 5 minutes or greater, 10 minutes or greater, 15 minutes or greater, 30 minutes or greater, 45 minutes or greater, 60 minutes or greater, 90 minutes or greater, 120 minutes or greater, and 240 minutes or greater.

Peripheral vasoconstriction is indicated in the methods described herein if the time difference (i.e., propagation time or transit time) between a feature in the central signal and the analogous feature in the peripheral signal is lower than a threshold value. This time lag is due to such features as arterial compliance and wave reflections in addition to others known to those of skill in the art. Example so threshold values useful with the methods as described herein include 100 milliseconds or fewer, 90 milliseconds or fewer, 80 milliseconds or fewer, 70 milliseconds or fewer, 60 milliseconds or fewer, 50 milliseconds or fewer, 40 milliseconds or fewer, or 30 milliseconds or fewer.

A cardiovascular condition also is indicated in the methods as described herein if there is a significant statistical change in the time difference between a feature in the central signal and the analogous feature in the peripheral signal over a specified time period. Examples of a significant statistical change useful with the methods as described herein include changes of 50 millisecond or greater, 60 milliseconds or greater, 70 milliseconds or greater, 80 milliseconds or greater, 90 milliseconds or greater, 100 milliseconds or greater, 110 milliseconds or greater, and 120 milliseconds or greater. Additional examples of a significant statistical change useful with the methods as described herein include changes of 0.4 standard deviations or greater, 0.5 standard deviations or greater, 0.6 standard deviations or greater, 0.7 standard deviations or greater, 0.8 standard deviations or greater, 0.9 standard deviations or greater, 1 standard deviations or greater, 1.5 standard deviations or greater, 2 standard deviations or greater, and 3 standard deviations or greater. Examples of threshold values useful with the methods as described herein include 5 minutes or greater, 10 minutes or greater, 15 minutes or greater, 30 minutes or greater, 45 minutes or greater, 60 minutes or greater, 90 minutes or greater, 120 minutes or greater, and 240 minutes or greater. Examples of specified time periods useful with the methods as described herein include 5 minutes or greater, 10 minutes or greater, 15 minutes or greater, 30 minutes or greater, 45 minutes or greater, 60 minutes or greater, 90 minutes or greater, 120 minutes or greater, and 240 minutes or greater.

The difference between a subject's central signal and peripheral signal or a statistical change over time in the difference can be continually monitored. Further, the difference between a subject's central signal and peripheral signal or a statistical change over time in the difference can be displayed on a graphical user interface. For example, the difference between the first signal and the second signal or a statistical change over time in the difference can be displayed as a bar graph or a trend graph. When a cardiovascular condition is detected, a user can be alerted, for example, by publishing a notice on a graphical user interface or by emitting a sound.

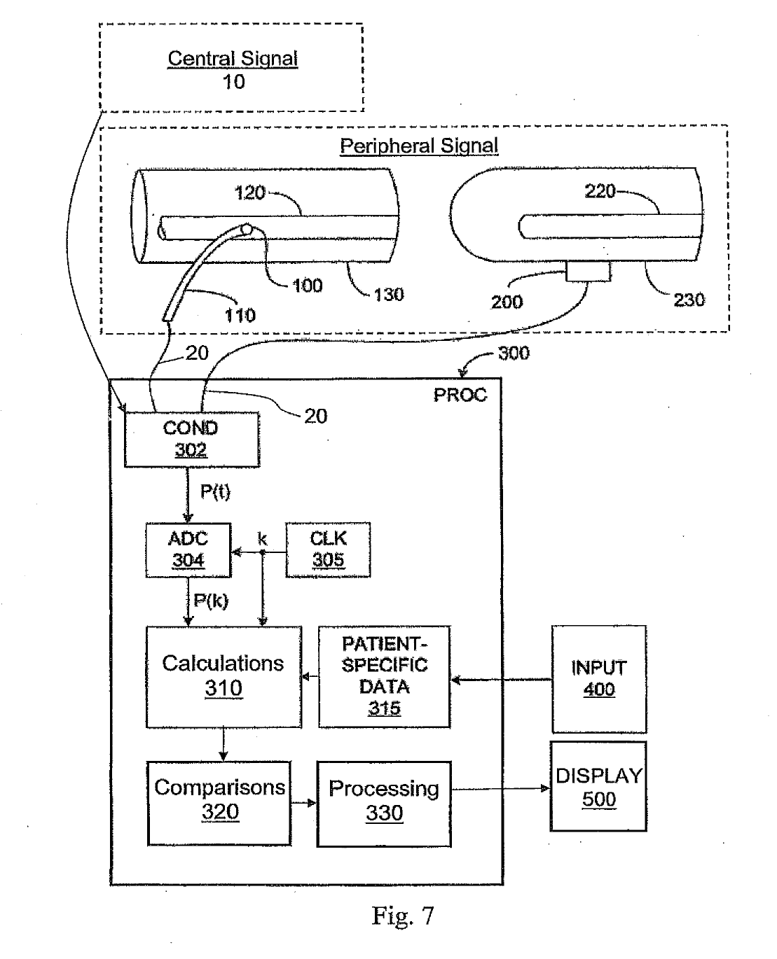

FIG. 7 shows the main components of a system that implements the methods described herein for monitoring a cardiovascular condition in a subject. The methods may be implemented within an existing patient-monitoring device, or it may be implemented as a dedicated monitor. As is mentioned above, a central signal proportional to or a function of the subject's heart activity (10) and a peripheral signal proportional to or a function of a signal related to the central signal (20), may be sensed in either or, indeed, both, of two ways: invasively and non-invasively.

As an example, FIG. 7 shows invasive and non-invasive techniques for measuring peripheral pressure and flow signals for this system. In most practical applications of the methods described herein, either one or several variations will typically be implemented for peripheral signal measurements. In invasive peripheral (or central) signal measurements for the methods described herein, a conventional pressure sensor or flow meter 100 is mounted on a catheter 110, which is inserted into or near an central or peripheral artery 120 of a portion 130 of the body of a human or animal subject. In the non-invasive applications of peripheral signal measurements for the methods described herein, a conventional pressure or flow sensor 200, such as a photo- or bioimpedance-plethysmographic blood pressure probe, is mounted externally in any conventional manner, for example using a cuff around a finger 230 or a transducer mounted on the wrist of the patient.

The signals from the sensors 100, 200 are passed via any known connectors as inputs to a processing system 300, which includes one or more processors and other supporting hardware and system software (not shown) usually included to process signals and execute code. The methods described herein may be implemented using a modified, standard, personal computer, or may be incorporated into a larger, specialized monitoring system. For use with the methods described herein, the processing system 300 also may include, or is connected to, conditioning circuitry 302 which performs normal signal processing tasks such as amplification, filtering, or ranging, as needed. The conditioned, sensed input signal is then converted to digital form by a conventional analog-to-digital converter ADC 304, which has or takes its time reference from a clock circuit 305. As is well understood, the sampling frequency of the ADC 304 should be chosen with regard to the Nyquist criterion so as to avoid aliasing of the pressure signal (this procedure is very well known in the art of digital signal processing). The output from the ADC 304 will be the discrete signal, whose values may be stored in conventional memory circuitry (not shown).

The signal values are passed to or accessed from memory by a software module 310 comprising computer-executable code for implementing one or more aspects of the methods as described herein. The design of such a software module 310 will be straight forward to one of skill in the art of computer programming. Additional comparisons and/or processing as used by a method can be performed in additional modules such as 320 and 330.

If used, signal-specific data such as a record of difference values or other calculations can be stored in a memory region 315, which may also store other data or parameters as needed. These values may be entered using any known input device 400 in the conventional manner.

As illustrated by FIG. 7, the results may be ultimately displayed on a conventional display or recording device 500 for presentation to and interpretation by a user. As with the input device 400, the display 500 will typically be the same as is used by the processing system for other purposes.

Exemplary embodiments of the present invention have been described above with reference to block diagrams and flowchart illustrations of methods, apparatuses, and computer program products. One of skill will understand that each block of the block diagrams and flowchart illustrations, and combinations of blocks in the block diagrams and flowchart illustrations, respectively, can be implemented by various means including computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

The methods described herein further relate to computer program instructions that may be stored in a computer-readable memory that can direct a computer or other programmable data processing apparatus, such as in a processor or processing system (shown as 300 in FIG. 7), to function in a particular manner, such that the instructions stored in the computer-readable memory produce an article of manufacture including computer-readable instructions for implementing the function specified in the blocks illustrated in FIG. 7. The computer program instructions may also be loaded onto a computer, the processing system 300, or other programmable data processing apparatus to cause a series of operational steps to be performed on the computer, the processing system 300, or other programmable apparatus to produce a computer-implemented process such that the instructions that execute on the computer or other programmable apparatus provide steps for implementing the functions specified in the blocks. Moreover, various software modules 310, 320, and 330 can be used to perform the various calculations and perform related method steps described herein also can be stored as computer-executable instructions on a computer-readable medium in order to allow the methods to be loaded into and executed by different processing systems.

Accordingly, blocks of the block diagrams and flowchart illustrations support combinations of means for performing the specified functions, combinations of steps for performing the specified functions, and program instruction means for performing the specified functions. One of skill will understand that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

The present invention is not limited in scope by the embodiments disclosed herein which are intended as illustrations of a few aspects of the invention and any embodiments which are functionally equivalent are within the scope of this invention. Various modifications of the methods in addition to those shown and described herein will become apparent to those skilled in the art and are intended to fall within the scope of the appended claims. Further, while only certain representative combinations of the method steps disclosed herein are specifically discussed in the embodiments above, other combinations of the method steps will become apparent to those skilled in the art and also are intended to fall within the scope of the appended claims. Thus a combination of steps may be explicitly mentioned herein; however, other combinations of steps are included, even though not explicitly stated. The term “comprising” and variations thereof as used herein is used synonymously with the term “including” and variations thereof and are open, non-limiting terms.

What is claimed is:

1. A method for monitoring a cardiovascular condition in a subject comprising:
measuring a central signal proportional to or a function of the subject's heart activity;
measuring a peripheral signal proportional to or a function of a signal related to the central signal; and
calculating a time difference between signal features representing the same heart events for the central signal and the peripheral signal,
wherein the cardiovascular condition is indicated if the time difference is greater than a threshold value.

2. A method for monitoring a cardiovascular condition in a subject comprising:
measuring a central signal proportional to or a function of the subject's heart activity;
measuring a peripheral signal proportional to or a function of a signal related to the central signal; and
calculating a time difference between signal features representing the same heart event for the central signal and the peripheral signal,
wherein the cardiovascular condition is indicated if there is a significant statistical change in the time difference over a specified time period.

3. The method of claim 1 or 2, wherein the cardiovascular condition is vasodilation.

4. The method of claim 1 or 2, wherein the cardiovascular condition is vasoconstriction.

5. The method of claim 1 or 2, wherein the cardiovascular condition indicates the occurrence of hyperdynamic circulation.

6. The method of claim 1 or 2, wherein the cardiovascular condition is central-to-peripheral pressure or flow decoupling.

7. The method of any of claim 3, 5, or 6, wherein the threshold value is 150 milliseconds or greater.

8. The method of claim 4, wherein the threshold value is 100 milliseconds or less.

9. The method of claim 2, wherein the statistically significant change is 50 milliseconds or greater.

10. The method of claim 2, wherein the statistically significant change is 0.4 standard deviations or greater.

11. The method of claim 1, wherein the threshold value is 5 minutes or greater.

12. The method of claim 2, wherein the specified time period is 5 minutes or greater.

13. The method of claim 1, wherein the cardiovascular condition is indicated is the time difference is greater than the threshold value for a specified time period.

14. The method of claim 13, wherein the specified time period is 5 minutes or greater.

15. The method of claim 1 or 2, wherein the central signal proportional to or a function of the subject's heart activity is one or more of electrocardiogram, aortic pressure, aortic flow, ultrasound, Doppler, transthoracic bioimpedance, or heart sounds.

16. The method of claim 1 or 2, wherein the peripheral signal proportional to or a function of a peripheral equivalent to the first signal is peripheral pressure, peripheral flow, ultrasound, Doppler, tonometry, or pulse oximetry.

17. The method of claim 1 or 2, wherein the central signal is aortic pressure and the peripheral signal is peripheral pressure.

18. The method of claim 1 or 2, wherein the central signal is aortic flow and the peripheral signal is peripheral flow.

19. The method of claim 1 or 2, wherein the central signal is an electrocardiogram signal and the peripheral signal is peripheral arterial pressure.

20. The method of claim 1, wherein the time difference between the central signal and the peripheral signal is continually monitored.

21. The method of claim 1, wherein the time difference between the central signal and the peripheral signal is displayed on a graphical user interface.

22. The method of claim 21, wherein the time difference between the central signal and the peripheral signal is displayed as a bar graph or a trend graph.

23. The method of claim 2, wherein the time difference between the central signal and the peripheral signal or a statistical change over time is continually monitored.

24. The method of claim 2, wherein the time difference between the central signal and the peripheral signal or a statistical change over time is displayed on a graphical user interface.

25. The method of claim 24, wherein the time difference between the central signal and the peripheral signal or a statistical change over time is displayed as a bar graph or a trend graph.

26. The method of claim 1, further comprising alerting a user when the cardiovascular condition is indicated.

27. The method of claim 2, further comprising alerting a user when the cardiovascular condition is indicated.

28. The method of claim 26 or 27, wherein the user is alerted by publishing a notice on a graphical user interface.

29. The method of claim 26 or 27, wherein the user is alerted by emitting a sound.
